(19) United States
(12) Patent Application Publication
(10) Pub. No.: US 2004/0101859 A1
(43) Pub. Date: May 27, 2004

(54) Compositions, methods and kits for polynucleotide amplification reactions and microfluidic devices

(75) Inventors: Byung Sook Moon, Palo Alto, CA (US); Jose Johnny Valdez, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Cepheid, Sunnyvale, CA

(21) Appl. No.: 10/305,229

(22) Filed: Nov. 25, 2002

(51) Int. Cl. C12Q 1/68; C12P 19/34; B01D 17/05

(52) U.S. Cl. 435/6; 435/91.2; 516/144

(57) ABSTRACT

Antifoam agents improve detection of polynucleotide amplification reactions and improve manipulation of fluids in microfluidic devices.

### BACKGROUND OF THE INVENTION

Detection of components of small amounts of fluids can be difficult for many reasons. One factor that can effect detection of very small volumes is bubbles. Moreover, manipulation of fluids in small areas, i.e., in microfluidic devices can lead to the development of bubbles. The present invention addresses these and other problems.

### BRIEF SUMMARY OF THE INVENTION

The present application provides reagent formulations for use in polynucleotide amplification and/or detection. In some embodiments, the reagents comprise at least one reagent for polynucleotide amplification or detection; and an antifoam agent.

In some embodiments, the antifoam agent is selected from the group consisting of a silicon-containing antifoam agent, organic sulfonate, polyether, fluorocarbon, organic phosphate, acetylenic glycol, polyisobutylene compound, poly (alkyl acrylate) compound, polyalkene polyamine, polyalkyleneimine compound and a blend thereof. In some embodiments, the reagent is an amplification mixture comprising a buffer; a disaccharide or disaccharide derivative; a carrier protein; and salt (e.g., MgCl₂).

In some embodiments, the buffer is HEPES.

In some embodiments, the formulation is an aqueous mixture. In some embodiments, the formulation is a solid.

In some embodiments, the reagent further comprises a DNA polymerase and deoxynucleotide triphosphates. In some embodiments, the DNA polymerase is Taq polymerase. In some embodiments, the reagent further comprises a polynucleotide template and at least one polynucleotide primer. In some embodiments, the reagent comprises a probe. In some embodiments, the probe is labeled with a fluorescent label.

In some embodiments, the formulation is an aqueous mixture and an active ingredient of the antifoam agent is in a concentration of 0.00001 g/ml to 0.0001 g/ml of the mixture.

In some embodiments, the antifoam agent contains silicon. In some embodiments, the antifoam agent contains silicone. In some embodiments, the antifoam agent is an organosiloxane polymer. In some embodiments, the antifoam agent is dimethylpolysiloxane. In some embodiments, the antifoam agent is Antifoam SE-15. In some embodiments, the formulation is an aqueous mixture and an active ingredient of SE-15 is in a concentration of 0.00001 to 0.0001 g/ml of the mixture.

In some embodiments, the antifoam agent does not contain silicon. In some embodiments, the antifoam agent does not contain silicone.

In some embodiments, the reagent comprises a dye that detects double-stranded DNA.

The present invention also provides a microfluidic device. In some embodiments, the microfluidic device contains a mixture, wherein the mixture comprises at least one reagent for amplifying or detecting a polynucleotide; and an antifoam agent.

In some embodiments, the antifoam agent is selected from the group consisting of a silicon-containing antifoam agent, organic sulfonate, polyether, fluorocarbon, organic phosphate, acetylenic glycol, polyisobutylene compound, poly (alkyl acrylate) compound, polyalkene polyamine, polyalkyleneimine compound and a blend thereof. In some embodiments, the reagent comprises a buffer; a disaccharide or disaccharide derivative; a carrier protein; deoxynucleotide triphosphates, a cation (e.g., Mg²⁺); a polynucleotide template; at least one polynucleotide primer; and a DNA polymerase.

In some embodiments, the buffer is HEPES. In some embodiments, the DNA polymerase is Taq polymerase.

In some embodiments, the antifoam agent is a silicon-based antifoam agent. In some embodiments, the antifoam agent is a silicone-based antifoam agent. In some embodiments, the antifoam agent is an organosiloxane polymer. In some embodiments, the antifoam agent is dimethylpolysiloxane. In some embodiments, the antifoam agent is Antifoam SE-15.

In some embodiments, the antifoam agent does not contain silicon. In some embodiments, the antifoam agent does not contain silicone.

In some embodiments, the mixture comprises a probe. In some embodiments, the probe is fluorescently labeled. In some embodiments, the mixture comprises a dye that binds to double-stranded DNA. In some embodiments, the antifoam in the mixture is in an amount such that an active ingredient of the antifoam agent is in a concentration of 0.00001 g/ml to 0.0001 g/ml of the mixture.

The present invention also provides a method of detecting the product of an amplification reaction. In some embodiments, the method comprises performing an amplification reaction in a mixture comprising an antifoam agent; and detecting the product of the amplification reaction.

In some embodiments, the antifoam agent is selected from the group consisting of a silicon-containing antifoam agent, organic sulfonate, polyether, fluorocarbon, organic phosphate, acetylenic glycol, polyisobutylene compound, poly (alkyl acrylate) compound, polyalkene polyamine, polyalkyleneimine compound and a blend thereof.

In some embodiments, the antifoam agent is a silicon-based antifoam agent. In some embodiments, the antifoam agent is an organosiloxane polymer. In some embodiments, the antifoam agent is dimethylpolysiloxane. In some embodiments, the antifoam agent is Antifoam SE-15.

In some embodiments, the antifoam agent does not contain silicon.

In some embodiments, the mixture further comprises HEPES.

In some embodiments, the amplification product is detected with a fluorescently-labeled probe. In some embodiments, the mixture is in a microfluidic device. In some embodiments, the antifoam in the mixture is in an amount such that an active ingredient of the antifoam agent is in a concentration of 0.00001 g/ml to 0.0001 g/ml of the mixture.

The present invention also provides methods of improving optical detection in a microfluidic device. In some embodiments, the methods comprise providing in the microfluidic device a mixture comprising an antifoam agent; and detecting a component of the mixture.

In some embodiments, the antifoam agent is selected from the group consisting of a silicon-containing antifoam agent, organic sulfonate, polyether, fluorocarbon, organic phosphate, acetylenic glycol, polyisobutylene compound, poly (alkyl acrylate) compound, polyalkene polyamine, polyalkyleneimine compound and a blend thereof.

In some embodiments, the antifoam agent is a silicon-based antifoam agent. In some embodiments, the antifoam agent is a silicone-based antifoam agent. In some embodiments, the antifoam agent is an organosiloxane polymer. In some embodiments, the antifoam agent is dimethylpolysiloxane. In some embodiments, the antifoam agent is Antifoam SE-15.

In some embodiments, the antifoam agent does not contain silicon. In some embodiments, the antifoam agent does not contain silicone.

In some embodiments, the antifoam in the mixture is in an amount such that an active ingredient of the antifoam agent is in a concentration of 0.00001 g/ml to 0.0001 g/ml of the mixture.

### DEFINITIONS

An “antifoam agent” as used herein refers to an agent that decreases or eliminates bubbles or foam in a mixture. Antifoam agents refer to agents that destroy existing stabilized foam and bubbles on the surface of liquid, prevent or retard the formation of foam or intensify bubble coalescence and accelerate foam release from liquid. The active ingredients of antifoam agents are often hydrophobic chemical substances, consequently the foam control agents are often insoluble in water. Foam control agents exist in water as hydrophobic fine droplets and adsorb or aggregate at air/liquid interfaces, i.e. bubble surfaces. The active ingredients of antifoams can have lower surface tension than that of foaming mediums and have a strong tendency to enter bubble films and spread across them, causing the bubbles to rupture. Antifoams can also bring a disorder into adsorption layers of surfactant molecules and destabilize the bubbles. Alternatively, antifoam agents can have lower surface tension than that of foaming mediums. Consequently, antifoam agents can enter bubble films and spread across them, causing the bubbles to rupture. In some cases, antifoam agents adsorb bubble surfaces like other surfactants in foaming liquids. After the bubbles come to the surfaces of foaming liquids, the bubble films become thin and rupture. In another alternative, antifoam agents adsorb bubble surfaces in liquids. In this case, antifoam agents accelerate entrapped small bubbles coalesce and grow bigger. Bigger bubbles rise faster to the liquid surfaces and rupture.

Antifoam agents are commonly also described as “defoamers” or “foam control agents” in the art. A number of standard tests for solution foaming have been described by the American Society of Testing and Materials (ASTM), including, e.g., a pour test (D1173-53), a shaking test (D3601-88) and a blending test (D3519-88). These tests can be used to determine the effectiveness of an antifoam agent. In some embodiments, at a particular concentration, an antifoam agent of the invention will typically reduce the quantity of foam in a solution, as tested by the ASTM tests listed above, by at least 10%, and sometimes by at least 25%, 50%, 75%, 90%, 95% or 99%. The concentration of the active ingredient of the antifoam agent will often be between 0.00001 and 0.0001 grams of active ingredient per milliliter of mixture. Those of skill in the art will recognize that the optimal concentration of antifoam agent will vary according to antifoam agent used, temperature, mixture, etc. In some embodiments, the concentration of the active ingredients is, e.g., about 0.00002 g/ml, 0.00003 g/ml, 0.00004 g/ml, 0.00005 g/ml, 0.00006 g/ml, 0.00007 g/ml, 0.00008 g/ml, or 0.00009 g/ml.

An “amplification reaction” refers to any chemical, including enzymatic, reaction that results in increased copies of a template nucleic acid sequence. Amplification reactions include polymerase chain reaction (PCR) and ligase chain reaction (LCR) (see U.S. Pat. Nos. 4,683,195 and 4,683,202; PCR Protocols: A Guide to Methods and Applications (Innis et al., eds, 1990)), strand displacement amplification (SDA) (Walker, et al. Nucleic Acids Res. 20(7):1691-6(1992); Walker PCR Methods Appl 3(1):1-6 (1993)), transcription-mediated amplification (Phyffer, et al., J Clin. Microbiol. 34:834-841 (1996); Vuorinen, et al., J. Clin. Microbiol. 33:1856-1859 (1995)), nucleic acid sequence-based amplification (NASBA) (Compton, Nature 350(6313):91-2 (1991), rolling circle amplification (RCA) (Lisby, Mol. BiotechnoL 12(1):75-99 (1999)); Hatch et al., Genet. Anal. 15(2):35-40 (1999)) and branched DNA signal amplification (bDNA) (see, e.g., Iqbal et al., Mol. Cell Probes 13(4):315-320 (1999)).

A “microfluidic device,” as used herein, refers to a device having one or more fluid passages, chambers or conduits which have at least one internal cross-sectional dimension, e.g., depth, width, length, diameter, etc., that is less than 1500 μm, and sometimes less than about 1000 μm, or about 500 μm, and typically between about 0.1 μm and about 500 μm.

The phrase “nucleic acid” or “polynucleotide” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

A “probe” refers to a polynucleotide sequence capable of hybridization to a polynucleotide sequence of interest and allows for the detecting of the polynucleotide sequence of choice. For example, “probes” can comprise polynucleotides linked to fluorescent or radioactive reagents, thereby allowing for the detection of these reagents. Examples of probes include fluorescently-labeled probes such as Taqman probes and molecular beacons.

A “reaction mixture” or “amplification reaction mixture,” as used herein, refers to either a mixture that can support amplification of a polynucleotide template without the addition of any other component or a mixture of a subset of the components required to amplify a template. For example, some components such as a DNA polymerase, may not be included in a reaction mixture so that the mixture can be stored under conditions that would degrade the enzyme prior to use. Similarly, for example, sequence-specific probes, primers, templates and/or nucleotides may, or may not, be included in a reaction mixture until amplification is to take place.

A “amplification reagent” or “reagent for polynucleotide amplification”, as used herein, refers to a reagent for use to amplify nucleic acids in an amplification reaction. The reagent can, but need not comprise all of the components required for an amplification reaction. Examples of components of an amplification reaction, can include, but are not limited to: nucleic acids, including templates, primers or deoxynucleotide triphosphates, a DNA polymerase (e.g., Taq polymerase), buffers (e.g., Tris, HEPES, etc.), salts such as magnesium and/or potassium-based salts, disaccharides or disaccharide derivatives, carrier proteins, detergents, DMSO, or other like agents.

A “reagent for detection” refers to any reagent containing a component to be detected or component, such as a probe or intercalating agent (e.g., ethidium bromide or SYBR Green), that assists in the detection of a component of a mixture. For example, the component to be detected can be a polynucleotide, protein or carbohydrate. Detection can be by, e.g., optical detection, e.g., using a photomultiplier or other instrumentation to detect fluorescence, radiation or other label.

A “thermocyclic amplification reaction” refers to the amplification of nucleic acid fragments by using primer oligonucleotides which, with the aid of a thermostable enzyme, synthesizes or ligates copies a template nucleic acid sequence. Thermocyclic reactions such as the polymerase chain reaction (PCR) and the ligase chain reaction (LCR) are well known.

A “target” or “target nucleic acid” refers to a single or double stranded polynucleotide sequence sought to be amplified in an amplification reaction.

A “template” refers to a double or single stranded polynucleotide sequence that comprises the polynucleotide to be amplified, flanked by primer hybridization sites.

### DETAILED DESCRIPTION OF THE INVENTION

I. Introduction

The present invention demonstrates for the first time that antifoam agents can be used in amplification reactions to improve detection of amplification products by reducing foaming that occurs, e.g., during mechanical agitation, including mixing, transferring, movement through tubing, dispensing, etc. Bubbles may also form during reagent thermal cycling, for example due to reagent degassing or by reaction of the liquid-plastic interface. Foam and bubble formation can block or interfere with the detection or transfer of reagent from one location to another, resulting in imprecision in amplification reaction results. The presence of an antifoam agent in the reaction mixture prevents or reduces these effects, thereby allowing for increased accuracy of measuring of the contents of a mixture. The antifoam agent is particularly helpful in the use of small volumes and in the detection of amplification products.

II. Antifoam Agents

Antifoam agents refer to any agent that prevents the development or speeds that breakdown of foam or bubbles in an aqueous mixture. A large number of antifoam agents are known to those of skill in the art and can comprise a number of different chemical structures. A number of different antifoam agents are described, for example, in Owens, “Defoamers” in ENCYCLOPEDIA OF CHEMICAL TECHNOLOGY (Kirk-Othmer, eds., 1993), pp928-945; Hofer et al., in ULLMANN'S ENCYCLOPEDIA OF INDUSTRIAL CHEMISTRY, (Elvers et al., eds., 1988) Vol. A11, 5th Ed, pp. 465-490; and Owen in ENCYCLOPEDIA OF POLYMER SCIENCE AND ENGINEERING, Vol. 2, 2nd. ed. (Kroschwitz, ed., 1987), pp59-72.

Antifoam compositions may comprise a single component or multiple components which may be combined by simply mixing together. However, some antifoam components are water-insoluble and thus some antifoam compositions may require mixing to produce the final antifoam composition.

In addition to active ingredients, antifoam agents can also include, e.g., carrier oils, amphiphilic substances and coupling or stabilizing agents. See, e.g., Hofer et al., in ULLMANN'S ENCYCLOPEDIA OF INDUSTRIAL CHEMISTRY, (Elvers et al., eds., 1988) Vol. A11, 5th Ed, pp. 465-490.

A. Silicon-based Antifoam Agents

A number of silicon-containing antifoam agents have been described. In some embodiments, silicon-based antifoams contain silicone. For example, organosiloxanes are a well-known class of silicon-based antifoam agents. Organosiloxanes include, e.g., pure silicone oils (such as dimethylpolysiloxanes) as well as polysiloxane/polyoxyalkylene block copolymers, including dimethylpolysiloxanes. These polymers may contain finely divided solids, which generally further promote the defoaming action. Examples of such finely divided solids are highly disperse, optionally hydrophobic, silicas obtained by pyrolysis or precipitation, magnesium or aluminum oxide as well as magnesium stearate.

In some embodiments, the antifoam agent is selected from Antifoam SE-15, Antifoam A, Antifoam B, Antifoam C, Antifoam SO-25, Antifoam SE-35 and Antifoam 289, each available from SIGMA. SE-15 is a 10% emulsion of active silicone polymer and non-ionic emulsifiers.

Other exemplary silicone-based antifoam agents are described in, e.g., Rosen, in U.S. Pat. No. 4,076,648, which teaches self-dispersible antifoam compositions consisting essentially of a lipophilic nonionic surface active agent homogeneously dispersed in a non-emulsified diorganopolysiloxane antifoam agent. This combination is said to promote dispersibility in water without the need for emulsification. Kulkarni et al., in U.S. Pat. No. 4,395,352, improved upon the compositions disclosed by Rosen, cited supra, by limiting the viscosity of the dimethylpolysiloxane oil therein to the range of 5,000 to 30,000 cS at 25° C. Such a limitation, it is taught, results in improved efficiency in difficult-to-defoam aqueous systems, such as those which contain high concentrations of ionic surfactants and those which are very viscous.

Keil, in U.S. Pat. No. 3,784,479, discloses foam control compositions which consist essentially of a base oil selected from polyoxypropylene polymers, polyoxypropylene-polyoxyethylene copolymers or siloxane-glycol copolymers, a foam control agent, comprising a liquid dimethylpolysiloxane and silica filler, and a dispersing agent which consists of a copolymer of a siloxane resin and a polyoxyalkylene polymer.

In a related patent, U.S. Pat. No. 3,984,347, Keil discloses foam control compositions which consist essentially of a base oil selected from polyoxypropylene polymers, polyoxypropylene-polyoxyethylene copolymers or siloxane-glycol copolymers, a foam control agent comprising a liquid dimethylpolysiloxane and silica filler and a siloxane copolymer dispersing agent.

John et al., in European Patent Application No. 217,501, published Apr. 8, 1987, discloses a foam control composition which gives improved performance in high foaming detergent compositions which comprises (A) a liquid siloxane having a viscosity at 25° C. of at least 7×10⁻³ m²/s and which was obtained by mixing and heating a triorganosiloxane-endblocked polydiorganosiloxane, a polydiorganosiloxane having at least one terminal silanol group and an organosiloxane resin, comprising monovalent and tetravalent siloxy units and having at least one silanol group per molecule, and (B) a finely divided filler having its surface made hydrophobic. John et al. further describes a method for making the foam control compositions and detergent compositions containing the foam control compositions.

Other silicone-based antifoam agents useful in the present invention are described in, e.g., U.S. Pat. Nos. 5,968,872 and 6,221,922 and European Patent Application Nos. EP 0046342 and 0791384. Antifoams comprising particulates, such as those described in U.S. Pat. No. 5,767,053 can also be used according to the present invention.

The concentration of any antifoam agent described herein will vary depending on the agent used. The concentration of active ingredients in antifoam agents can vary greatly. For example, in some cases, the concentration of an active ingredient in a mixture will be from 0.000001 g/ml to 0.1 g/ml and sometimes between 0.00001 g/ml to 0.0001 g/ml. In amplification reactions comprising silicone-based antifoams, such as Antifoam SE-15, the concentration of the antifoam active ingredient can be, e.g., between 0.00001 g/ml to 0.0001 g/ml. A preferred concentration is 0.00006 g/ml (equal to 0.00006 mg/μl, 0.06 μg/μl or 60 ng/μl) weight/volume. For example, in some embodiments, the antifoam preparations are prepared with 0.006 g/100 ml of the mixture. In some embodiments, the mixture is aliquoted into 25, 50 or 100 μl volumes for amplification.

Silicone-based antifoam agents can be provided in an emulsifying formulation, e.g., with a non-ionic emulsifier, to prevent aggregation of the antifoam agent in an aqueous mixture. Thus, in some embodiments, the silicone-based antifoam agent forms an emulsion in the amplification reaction or detection mixture.

B. Non-Silcon-based Antifoam Agents

Non-silicon antifoam agents can be any antifoam agent that does not contain silicon. Representative examples include, e.g., hydrocarbons such as, e.g., organic sulfonates, polyethers, organic phosphates, acetylenic glycols, polyisobutylene compounds, poly (alkyl acrylate) compounds, polyalkene polyamines, and polyalkyleneimine compounds. Other antifoam agents include fluorocarbons. Organic sulfonates, carbon powders, vegetable oils, animal oils, polyisobutylene compounds and blends thereof are disclosed in, e.g., U.S. Pat. Nos. 5,169,560; 5,296,132; 5,389,299; and 5,472,637. Other representative non-silicon containing antifoam agents are described in, e.g., Hofer et al., in ULLMANN'S ENCYCLOPEDIA OF INDUSTRIAL CHEMISTRY, (Elvers et al., eds., 1988) Vol. A11, 5th Ed, pp. 465-490; and Owen in ENCYCLOPEDIA OF POLYMER SCIENCE AND ENGINEERING, Vol. 2, 2nd. ed. (Kroschwitz, ed., 1987), pp59-72. These compounds are given by way of example and should not be construed as limiting the non-silicone defoamer compounds that can be used in this invention.

III. Amplification Reactions

A. General Information

The present invention provides various compositions and reaction mixtures for use in amplification reactions and microfluidic devices. In general, the invention provides amplification mixtures that comprise antifoam agents, such as those exemplified herein. Those of skill in the art will recognize, however, that reaction mixtures and components thereof, can be in either liquid or solid form. For example, lyophilized mixtures are often stored before use and can be incorporated into kits. See, e.g., U.S. Pat. Nos. 5,834,254; 5,876,992; and 6,153,412. In some cases, the solid mixtures can be incorporated into beads or “spheres.” See, e.g., U.S. Pat. No. 5,593,824.

Reaction mixtures can, but need not have all components required to complete an amplification reaction. For example, in some circumstances it is convenient to store a mixture of some, but not all of the components required for an amplification reaction. In some cases, all components but the nucleic acids are in the mixture. In some embodiments, only the components that are stable at room temperature or in a lyophilized mixture are included in the mixture.

Thus, in some aspects, the amplification mixture comprises a buffer, a disaccharide or disaccharide derivative, a carrier protein, magnesium, and an antifoam agent. In some aspects, the reaction mixture also comprises a DNA polymerase such as Taq polymerase and/or deoxynucleotide triphosphates (e.g., dATP, dCTP, dTTP, dGTP). In some embodiments, the reaction mixtures of the invention will lack a template polynucleotide, primers or a probe.

In some aspects, the reaction mixtures are contained in a microfluidic device. For example, because of the presence of the antifoam agent, optical detection of components in the device is greatly improved. This improvement is particularly useful in a microfluidic device where bubbles can disrupt the very small volumes of liquid within. The antifoam prevents foam which can interfere with optical detection. Exemplary microfluidic devices that employ amplification reaction mixtures include, e.g., the SmartCycler®, GeneXpert® and I-CORE® devices (Cepheid, Sunnyvale, Calif.). However, the advantages of the mixtures of the invention extend to all microfluidic devices, whether for use in amplification reactions or not. For example, transfer of small amounts of fluids in such devices can be inhibited by the presence of bubbles in the mixtures. The mixtures of the present invention address this problem.

Amplification of an RNA or DNA template using reaction mixtures is well known (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences of target DNA sequences directly from, e.g., mRNA, from cDNA, from genomic libraries or cDNA libraries as well as from organisms, environmental samples, or any other source of nucleic acids. The reaction is preferably carried out in a thermal cycler to facilitate incubation times at desired temperatures.

Exemplary PCR reaction conditions typically comprise either two or three step cycles. Two step cycles have a denaturation step followed by a hybridization/elongation step. Three step cycles comprise a denaturation step followed by a hybridization step followed by a separate elongation step.

Isothermic amplification reactions are also known and can be used according to the methods of the invention. Examples of isothermic amplification reactions include strand displacement amplification (SDA) (Walker, et al. Nucleic Acids Res. 20(7):1691-6 (1992); Walker PCR Methods Appl 3(1):1-6 (1993)), transcription-mediated amplification (Phyffer, et al., J. Clin. Microbiol. 34:834-841 (1996); Vuorinen, et al. , J Clin. Microbiol. 33:1856-1859 (1995)), nucleic acid sequence-based amplification (NASBA) (Compton, Nature 350(6313):91-2 (1991), rolling circle amplification (RCA) (Lisby, Mol. Biotechnol. 12(1):75-99 (1999)); Hatch et al., Genet. Anal. 15(2):35-40 (1999)) and branched DNA signal amplification (bDNA) (see, e.g., Iqbal et al., Mol. Cell Probes 13(4):315-320 (1999)). Other amplification methods known to those of skill in the art include CPR (Cycling Probe Reaction), SSR (Self-Sustained Sequence Replication), SDA (Strand Displacement Amplification), QBR (Q-Beta Replicase), Re-AMP (formerly RAMP), RCR (Repair Chain Reaction), TAS (Transcription Based Amplification System), and HCS.

B. Amplification Reaction Components

The mixtures of the invention can comprises any or all of the following amplification reaction mixture components. Those of skill in the art will recognize that a number of amplification reagents have been described in the art. The list below is not comprehensive.

Oligonucleotide Primers

The oligonucleotides that are used in the present invention as well as oligonucleotides designed to detect amplification products can be chemically synthesized. These oligonucleotides can be labeled with radioisotopes, chemiluminescent moieties, or fluorescent moieties. Such labels are useful for the characterization and detection of amplification products using the methods and compositions of the present invention.

The primer components may be present in the PCR reaction mixture at a concentration of, e.g., between 0.1 and 1.0 μM. The primer length can be between, e.g., 8-100 nucleotides in length and preferably have 50-60% G and C composition. In the choice of primer, it is preferable to have exactly matching bases at the 3′ end of the primer but this requirement decreases to relatively insignificance at the 5′ end. In some embodiments, the primers of the invention all have approximately the same melting temperature.

Buffer

Exemplary buffers that may be employed, include, e.g., HEPES, borate, phosphate, carbonate, barbital, Tris, etc. based buffers. See Rose et al., U.S. Pat. No. 5,508,178. The pH of the reaction should be maintained in the range of about 4.5 to about 9.5. See U.S. Pat. No. 5,508,178. The standard buffer used in amplification reactions is a Tris based buffer between 10 and 50 mM with a pH of around 8.3 to 8.8. See Innis et al., supra.

One of skill in the art will recognize that buffer conditions should be designed to allow for the function of all reactions of interest. Thus, buffer conditions can be designed to support the amplification reaction as well as any enzymatic reactions associated with producing signals from probes. A particular reaction buffer can be tested for its ability to support various reactions by testing the reactions both individually and in combination.

Salt Concentration

The concentration of salt present in the reaction mixture can affect the ability of primers to anneal to the target nucleic acid. See Innis et al. Potassium chloride is typically added up to a concentration of about 50 mM or more to the reaction mixture to promote primer annealing. Sodium chloride can also be added to promote primer annealing. See Innis et al.

Magnesium Ion Concentration

The concentration of magnesium ion in the reaction can be critical to amplifying the desired sequence(s). See Innis et al. Primer annealing, strand denaturation, amplification specificity, primer-dimer formation, and enzyme activity are all examples of parameters that are affected by magnesium concentration. See Innis et al. Amplification reactions can contain, e.g., about a 0.5 to 2.5 mM magnesium concentration excess over the concentration of dNTPs. The presence of magnesium chelators in the reaction can affect the optimal magnesium concentration. A series of amplification reactions can be carried out over a range of magnesium concentrations to determine the optimal magnesium concentration. The optimal magnesium concentration can vary depending on the nature of the target nucleic acid(s) and the primers being used, among other parameters. A common source of magnesium ion is MgCl₂.

Disaccharide or Disaccharide Derivatives

Exemplary disaccharides useful in the present invention include, but are not limited to, trehalose, sucrose, and others. Exemplary disaccharide derivatives useful in the present invention include maltitol, mannitol, branched sucrose polymers, for example FICOLL®, sorbitol, and others, such as disaccharide alcohols.

Exemplary sugar polymers useful in the present invention include dextran.

Carrier Proteins

Carrier proteins useful in the present invention include but are not limited to albumin (e.g., bovine serum albumin) and gelatin.

Deoxynucleotide Triphosphate Concentration

Deoxynucleotide triphosphates (dNTPs) is added to the reaction to a final concentration of about 20 μM to about 300 μM. Each of the four dNTPs (G, A, C, T) are generally present at equivalent concentrations. See Innis et al.

Nucleic Acid Polymerase

A variety of DNA dependent polymerases are commercially available that will function using the methods and compositions of the present invention. For example, Taq DNA Polymerase may be used to amplify target DNA sequences. The PCR assay may be carried out using as an enzyme component a source of thernostable DNA polymerase suitably comprising Taq DNA polymerase which may be the native enzyme purified from Thermus aquaticus and/or a genetically engineered form of the enzyme. Other commercially available polymerase enzymes include, e.g., Taq polymerases marketed by Promega or Pharmacia. Other examples of thermostable DNA polymerases that could be used in the invention include DNA polymerases obtained from, e.g., Thermus and Pyrococcus species. Concentration ranges of the polymerase may range from 1-5 units per reaction mixture. The reaction mixture is typically between 20 and 100 μl.

In some embodiments, a “hot start” polymerase can be used to prevent extension of mispriming events as the temperature of a reaction initially increases. Hot starts are particularly useful in the context of multiplex PCR. Hot start polymerases can have, for example, heat labile adducts requiring a heat activation step (typically 95° C. for approximately 10-15 minutes) or can have an antibody associated with the polymerase to prevent activation.

Other Agents

Assorted other agents are sometime added to the reaction to achieve the desired results. For example, DMSO can be added to the reaction, but is reported to inhibit the activity of Taq DNA Polymerase. Nevertheless, DMSO has been recommended for the amplification of multiple target sequences in the same reaction. See Innis et al. Non-ionic detergents (e.g. Tween-20) can also be added to amplification reactions. See Innis et al. In addition, methyisothiazolin can be added to the reaction mixture.

IV. Kits

The invention also provides kits for carrying out the amplification methods of the invention. For example, the invention provides kits that include one or more reaction vessels that have aliquots of some or all of the reaction mixture components of the invention in them. Aliquots can be in liquid or dried form. Reaction vessels can include sample processing cartridges or other vessels that allow for the containment, processing and/or amplification of samples in the same vessel. Such kits allow ready detection of amplification products into standard or portable amplification devices. The kits can also include written instructions for the use of the kit to amplify and control for amplification of a target sample.

Kits can include, for instance, a reagent for polynucleotide amplification or detection and an antifoam agent. The kit can contain one or more probes (e.g. Taqmang or molecular beacon probes) comprising a fluorophore, and optionally, a quenching agent. In addition, the kit can include nucleotides (A, C, G, T) and a DNA polymerase.

In some embodiments, the kits comprise a microfluidic device. For example, vessels such as sample processing cartridges useful for rapid amplification of a sample as described in Belgrader, P., et al., Biosensors and Bioelectronics 14:849-852 (2000); Belgrader, P., et al., Science, 284:449-450 (1999); and Northrup, M. A., et al. “A New Generation of PCR Instruments and Nucleic Acid Concentration Systems” in PCR PROTOCOLS (Sninsky, J. J. et al (eds.)) Academic, San Diego, Chapter 8 (1998)) can be included in the kits of the invention.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A reagent formulation, comprising at least one reagent for polynucleotide amplification or detection; and
an antifoam agent.

2. The formulation of claim 1, wherein the antifoam agent is selected from the group consisting of a silicon-containing antifoam agent, organic sulfonate, polyether, fluorocarbon, organic phosphate, acetylenic glycol, polyisobutylene compound, poly (alkyl acrylate) compound, polyalkene polyamine, polyalkyleneimine compound and a blend thereof.

3. The formulation of claim 1, wherein the reagent is an amplification mixture comprising
a buffer;
a disaccharide or disaccharide derivative;
a carrier protein; and
a salt.

4. The formulation of claim 3, wherein the buffer is HEPES.

5. The formulation of claim 1, wherein the formulation is an aqueous mixture.

6. The formulation of claim 1, wherein the formulation is a solid.

7. The formulation of claim 3, wherein the reagent further comprises a DNA polymerase and deoxynucleotide triphosphates.

8. The formulation of claim 7, wherein the DNA polymerase is Taq polymerase.

9. The formulation of claim 7, wherein the reagent further comprises a polynucleotide template and at least one polynucleotide primer.

10. The formulation of claim 1, wherein the reagent comprises a probe.

11. The formulation of claim 17, wherein the probe is labeled with a fluorescent label.

12. The formulation of claim 1, wherein the formulation is an aqueous mixture and an active ingredient of the antifoam agent is in a concentration of 0.00001 g/ml to 0.0001 g/ml of the mixture.

13. The formulation of claim 1, wherein the antifoam agent contains silicon.

14. The formulation of claim 13, wherein the antifoam agent contains silicone.

15. The formulation of claim 1, wherein the antifoam agent is an organosiloxane polymer.

16. The formulation of claim 1, wherein the antifoam agent is dimethylpolysiloxane.

17. The formulation of claim 1, wherein the antifoam agent is Antifoam SE-15.

18. The formulation of claim 17, wherein the formulation is an aqueous mixture and an active ingredient of SE-15 is in a concentration of 0.00001 to 0.0001 g/ml of the mixture.

19. The formulation of claim 1, wherein the antifoam agent does not contain silicon.

20. The formulation of claim 1, wherein the reagent comprises a dye that detects double-stranded DNA.

21. A microfluidic device containing a mixture, wherein the mixture comprises
at least one reagent for amplifying or detecting a polynucleotide; and
an antifoam agent.

22. The microfluidic device of claim 21, wherein the antifoam agent is selected from the group consisting of a silicon-containing antifoam agent, organic sulfonate, polyether, fluorocarbon, organic phosphate, acetylenic glycol, polyisobutylene compound, poly (alkyl acrylate) compound, polyalkene polyamine, polyalkyleneimine compound and a blend thereof.

23. The microfluidic device of claim 21, wherein the reagent comprises
a buffer;
a disaccharide or disaccharide derivative;
a carrier protein;
deoxynucleotide triphosphates
a cation;
a polynucleotide template;
at least one polynucleotide primer; and
a DNA polymerase.

24. The microfluidic device of claim 23, wherein the buffer is HEPES.

25. The microfluidic device of claim 23, wherein the DNA polymerase is Taq polymerase.

26. The microfluidic device of claim 21, wherein the antifoam agent is a silicon-based antifoam agent.

27. The microfluidic device of claim 21, wherein the antifoam agent is a silicone-based antifoam agent.

28. The microfluidic device of claim 21, wherein the antifoam agent is an organosiloxane polymer.

29. The microfluidic device of claim 21, wherein the antifoam agent is dimethylpolysiloxane.

30. The microfluidic device of claim 21, wherein the antifoam agent is Antifoam SE-15.

31. The microfluidic device of claim 21, wherein the antifoam agent does not contain silicon.

32. The microfluidic device of claim 31, wherein the mixture comprises a probe.

33. The microfluidic device of claim 32, wherein the probe is fluorescently labeled.

34. The microfluidic device of claim 31, wherein the mixture comprises a dye that binds to double-stranded DNA.

35. The microfluidic device of claim 21, wherein the antifoam in the mixture is in an amount such that an active ingredient of the antifoam agent is in a concentration of 0.00001 g/ml to 0.0001 g/ml of the mixture.

36. A method of detecting the product of an amplification reaction, the method comprising,
performing an amplification reaction in a mixture comprising an antifoam agent; and
detecting the product of the amplification reaction.

37. The method of claim 36, wherein the antifoam agent is selected from the group consisting of a silicon-containing antifoam agent, organic sulfonate, polyether, fluorocarbon, organic phosphate, acetylenic glycol, polyisobutylene compound, poly (alkyl acrylate) compound, polyalkene polyamine, polyalkyleneimine compound and a blend thereof.

38. The method of claim 36, wherein the antifoam agent is a silicon-based antifoam agent.

39. The method of claim 36, wherein the antifoam agent is a silicone-based antifoam agent.

40. The method of claim 36, wherein the antifoam agent is an organosiloxane polymer.

41. The method of claim 36, wherein the antifoam agent is dimethylpolysiloxane.

42. The method of claim 36, wherein the antifoam agent is Antifoam SE-15.

43. The method of claim 36, wherein the antifoam agent does not contain silicon.

44. The method of claim 36, wherein the mixture further comprises HEPES.

45. The method of claim 36, wherein the amplification product is detected with a fluorescently-labeled probe.

46. The method of claim 36, wherein the mixture is in a microfluidic device.

47. The method of claim 36, wherein the antifoam in the mixture is in an amount such that an active ingredient of the antifoam agent is in a concentration of 0.00001 g/ml to 0.0001 g/ml of the mixture.

48. A method of improving optical detection in a microfluidic device, the method comprising,
providing in the microfluidic device a mixture comprising an antifoam agent; and
detecting a component of the mixture.

49. The method of claim 48, wherein the antifoam agent is selected from the group consisting of a silicon-containing antifoam agent, organic sulfonate, polyether, fluorocarbon, organic phosphate, acetylenic glycol, polyisobutylene compound, poly (alkyl acrylate) compound, polyalkene polyamine, polyalkyleneimine compound and a blend thereof.

50. The method of claim 48, wherein the antifoam agent is a silicon-based antifoam agent.

51. The method of claim 48, wherein the antifoam agent is a silicone-based antifoam agent.

52. The method of claim 48, wherein the antifoam agent is an organosiloxane polymer.

53. The method of claim 48, wherein the antifoam agent is dimethylpolysiloxane.

54. The method of claim 48, wherein the antifoam agent is Antifoam SE-15.

55. The method of claim 48, wherein the antifoam agent does not contain silicon.

56. The method of claim 48, wherein the antifoam in the mixture is in an amount such that an active ingredient of the antifoam agent is in a concentration of 0.00001 g/ml to 0.0001 g/ml of the mixture.
